(19) United States
(12) Patent Application Publication
(10) Pub. No.: US 2022/0208373 A1
(43) Pub. Date: Jun. 30, 2022

(54) INQUIRY RECOMMENDATION FOR MEDICAL DIAGNOSIS

(71) Applicant: INTERNATIONAL BUSINESS MACHINES CORPORATION, Armonk, NY (US)

(72) Inventors: Ya Bin Dang, Beijing (CN); Ke Wang, Beijing (CN); Jian Wang, Beijing (CN); JIAN MIN JIANG, Beijing (CN)

(21) Appl. No.: 17/139,014

(22) Filed: Dec. 31, 2020

(51) Int. Cl. G16H 50/20; G06N 5/02; G06F 40/30

(52) U.S. Cl. CPC G16H 50/20; G06F 40/30; G06N 5/022

(57) ABSTRACT

A machine-guided inquiry recommendation for medical diagnosis. Generate a knowledge graph data structure using one or more electronic medical guideline documents. Evaluate a likelihood value for one or more diseases based on the knowledge graph data structure. Generate a best next inquiry question for use in a medical diagnosis process.

### BACKGROUND

Embodiments of the invention generally relate to computing systems and methods for enhancing inquiry recommendations for medical diagnosis.

Advances in computer science continue to improve computerized healthcare technology by augmenting, for example, means by which a user performs medical diagnosis. Given a set of input data, a computerized healthcare solution may calculate probabilities that the input data indicates one or more diseases. The solution may, for example, employ a variety of tools and techniques to provide a medical professional with a list of suspected diseases (based on the input data), where the list is generated using a knowledge graph; and to assist the medical professional in generating inquiries that allow the medical professional to narrow in on a correct diagnosis based on the list.

### SUMMARY

Some embodiments of the invention provide for methods, computer program products, and systems for a machine-guided inquiry recommendation. The embodiments generate a best next inquiry question, for use in a medical diagnosis process. The generating is based on one or more likelihood values that a set of input symptoms exhibits one or more diseases. The likelihood values are calculated using a knowledge graph data structure generated using one or more electronic medical guideline documents.

In an embodiment, generating the knowledge graph data structure is performed using one or more electronic medical guideline documents.

In an embodiment, calculating the one or more likelihood values comprises applying a combined term frequency-inverse document frequency (TF-IDF) process and Bayesian modelling process to one or more diseases in the knowledge graph data structure. In an embodiment, the applying comprises: applying the combined TF-IDF process and Bayesian modeling process to obtain an acceptable performance value; optimizing weight values of symptoms used in the applying; and applying further Bayesian modelling to improve accuracy of the one or more likelihood values.

In an embodiment, generating a best next inquiry is performed using an index.

In an embodiment, calculating the one or more likelihood values comprises: performing feature recognition using an object-oriented symptom modeling process; performing feature selection on recognized features to infer possible features; performing feature prioritization on selected features using an information retrieval process; performing a similarity analysis on prioritized features to calculate distance of prioritized features; and generating a list of suspected diseases based on the similarity analysis.

Some embodiments of the invention provide for methods, computer program products, and systems for providing a machine-guided inquiry recommendation for medical diagnosis. The embodiments detect a set of factual nodes in a knowledge graph data structure; infer a set of evidential nodes in the set of factual nodes; infer a set of disease feature nodes in the set of evidential nodes; and infer a set of possible disease nodes in the set of diseases feature nodes.

In an embodiment, detecting a set of factual nodes in a knowledge graph data structure comprises: measuring, using semantic matching, a similarity between an input text one or more nodes in the knowledge graph data structure, the input text comprising one or more observed symptoms, wherein the measuring yields a confidence score.

In an embodiment, inferring a set of evidential nodes in the set of factual nodes comprises: identifying one or more candidate evidential nodes among the set of factual nodes, wherein the identifying is based on hyponymy relationships between at least two factual nodes in the set of factual nodes and is further based on a confidence value for each of the one or more candidate evidential nodes; adding to the set of inferred set of evidential nodes, at least one of the one or more candidate evidential nodes based on its corresponding confidence value, the at least one factual node as an inferred evidential node.

In an embodiment, inferring a set of disease feature nodes in the set of evidential nodes comprises: determining a set of disease feature nodes in the set of evidential nodes; and inferring a set of possible disease nodes in the set of diseases feature nodes.

### BRIEF DESCRIPTION OF THE SEVERAL VIEWS OF THE DRAWINGS

FIG. 1A is a block diagram of a first illustrative symptom-disease knowledge graph 100A, according to an embodiment of the invention.

FIG. 1B is a second illustrative symptom-disease knowledge graph 100B, according to an embodiment of the invention.

FIG. 1C is a Venn diagram 100C of an illustrative sets definition, according to an embodiment of the invention.

FIG. 2 is a block diagram of an illustrative symptom-disease knowledge graph 200, according to an embodiment of the invention.

FIG. 3 depicts an illustrative process for calculating a suspected disease using a term-frequency-inverse document frequency (TF-IDF) approach.

FIG. 4 is a flowchart of a method 400 for calculating a suspected disease using a symptom-disease knowledge graph and a corpus of medical guideline documents (for example symptom-disease knowledge graph 110B of FIG. 1 or symptom-disease knowledge graph 200 of FIG. 2), according to an embodiment of the invention.

FIG. 5 is a functional flow diagram of a method 500 for calculating a suspected disease using a symptom-disease knowledge graph and a corpus of medical guideline documents (for example symptom-disease knowledge graph 110B of FIG. 1 or symptom-disease knowledge graph 200 of FIG. 2), according to an embodiment of the invention.

FIG. 6 describes an approach 600 for inferring suspected disease nodes (for example, as described in FIG. 5, above), according to an embodiment of the invention.

FIG. 7 describes an approach 700 for generating an effective inquiry based on the Gini index, according to an embodiment of the invention.

FIG. 8 is a block diagram of an illustrative cloud computing node, according to an embodiment of the invention.

FIG. 9 is a block diagram of a cloud computing environment including the cloud computing node of FIG. 8, according to an embodiment of the invention.

FIG. 10 is a block diagram of functional layers of the cloud computing environment of FIG. 9, according to an embodiment of the invention.

### DETAILED DESCRIPTION

The combination of the computerized systems and methods of their use that are disclosed as embodiments of the invention enable medical professionals to perform at least some tasks, identify at least some existing information, and to obtain at least some new information, that the medical professionals cannot do mentally or by pen and paper alone, without the aid of computerized technology. As such, embodiments of the invention constitute improvements to computing technology itself. The claimed embodiments of the invention do not seek to protect any process of medical diagnosis independently of the computing technology that is disclosed or claimed. More specifically, embodiments of the invention are directed to providing a machine-guided inquiry recommendation for medical diagnosis; and not to inquiry recommendation for medical diagnosis.

Generally, computerized medical diagnosis can be improved by determining, for a given set of symptoms, probabilities that the set of symptoms indicate one or more diseases. For example, for a set of symptoms S, and a set of diseases D, a probability may be calculated for each particular disease in D, representing the likelihood that a patient exhibiting the symptoms in S suffers from that particular disease. In an embodiment, this process may include generating a symptom-disease knowledge graph by: receiving as input one or more electronic documents constituting medical guidelines; extracting a set of diseases and symptoms from the medical guidelines, where the diseases may be related to one another in a hierarchy or other relational manner; and generating models of the relations among symptoms and diseases based on the extracted information. These steps may be performed using, for example, electronic natural language processing systems and methods. The symptom-disease knowledge graph can be used to generate or guide inquiry recommendations for a more targeted medical diagnosis, for example by identifying a suspect disease list using known symptoms and the symptom-disease knowledge graph, and by generating an inquiry question to guide the diagnosis. In an embodiment, these functions may be incorporated into an electronic question and answering (QA) system, a chat bot system, or another system, and trained using machine learning and other artificial intelligence paradigms.

Embodiments of the invention will now be described in connection with the Figures.

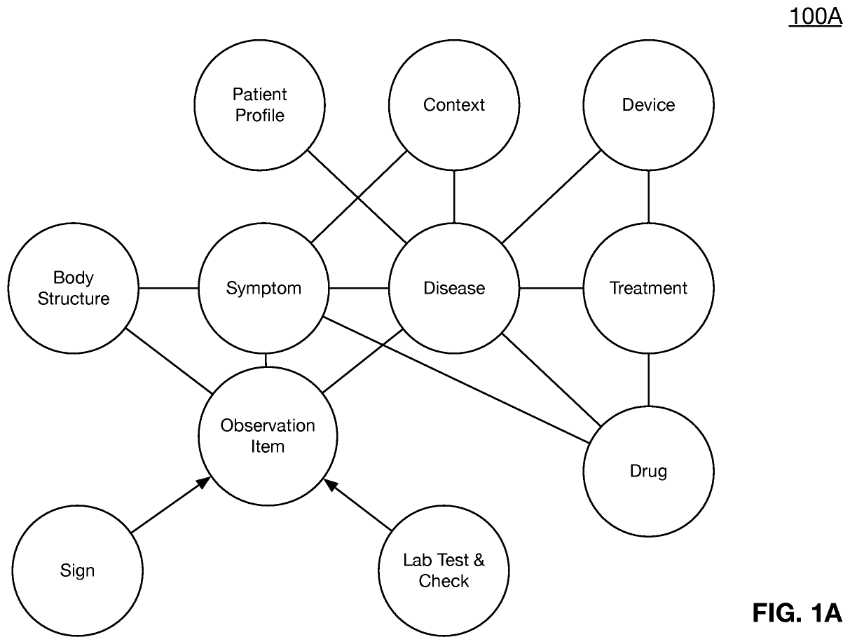

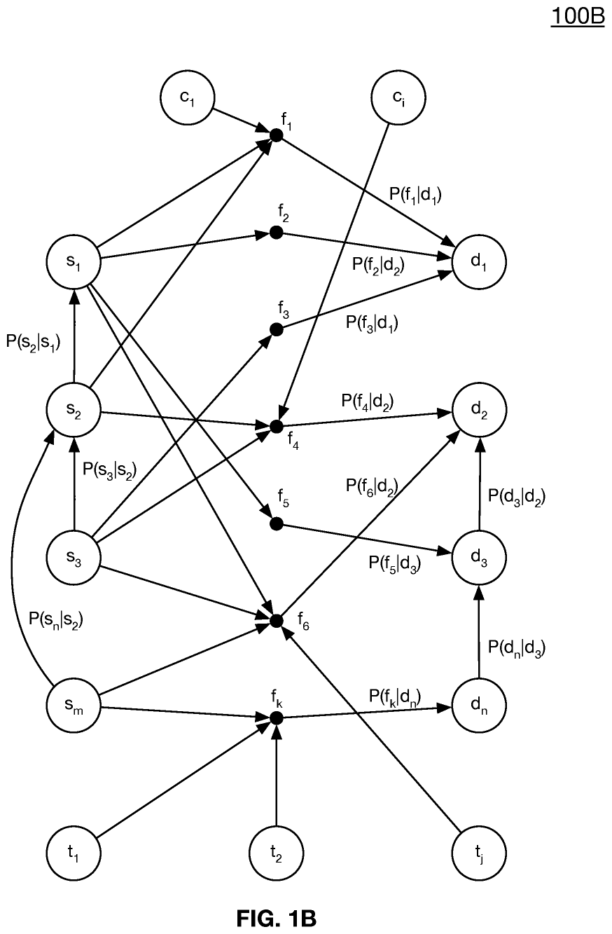

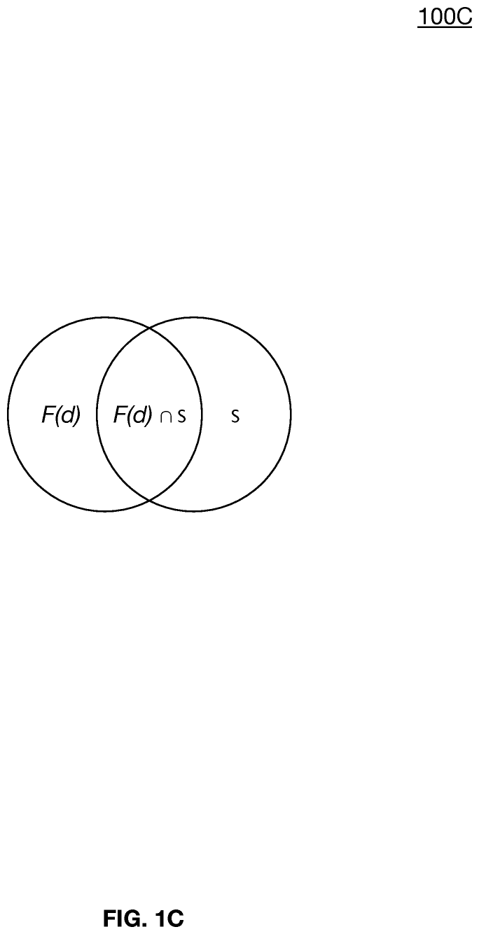

FIG. 1A is a block diagram of a first illustrative symptom-disease meta-model knowledge graph 100A, according to an embodiment of the invention. Symptom-disease meta-model knowledge graph 100A may be generated according to any known method in the art, including through processes described above. In this example, symptom-disease meta-model knowledge graph 100A includes a set of nodes and edges; the nodes include a disease node, a symptom node, a body structure node, a context node, a device node, a treatment node, a drug node, an observation item node, a sign node, a lab test and check node, and a patient profile node. The nodes may be connected according to any known method in the art where, using natural language processing and other techniques, relations are identified as between the entities or concepts denoted by two nodes in the graph. Directional edges indicate a flow of information from one node to another. It shall be understood that the specific nodes depicted in FIG. 1 are provided for illustration, and symptom-disease meta-model knowledge graph 100A need not include all these nodes (and may include other nodes) and need not store their corresponding information in this specific structure.

FIG. 1B is a block diagram of a first illustrative symptom-disease meta-model knowledge graph 100B, according to an embodiment of the invention. Symptom-disease meta-model knowledge graph 100B includes the following three sets of nodes: a set of factual nodes (symptom nodes, test nodes and context nodes), a set of feature nodes, and a set of disease nodes.

In the depicted embodiment, the factual nodes include symptom nodes {s₁, s₂, s₃ . . . sₘ}; context nodes {c₁ . . . cᵢ}, and test nodes {t₁, t₂ . . . tⱼ}. The feature nodes include {f₁, f₂, f₃, f₄, f₅, f₆ . . . fₖ}. The disease nodes include {d₁, d₂, d₃, . . . , dₙ}.

In the depicted embodiment, each symptom node may be connected to one or more other symptom nodes and to one or more features. Each connection to another symptom has a corresponding conditional probability P. For example, in the depicted embodiment, Si has P(s₂|s₁); s₂ has P(s₃|s₂) and P(sₘ|s₂). A given conditional probability P(sₘ₁|sₘ₂) indicates, for a pair of symptoms (sₘ₁, sₘ₂), the likelihood of the symptom sₘ₁ occurring when the symptom sₘ₂ is observed.

In the depicted embodiment, each disease node is connected to one or more feature nodes and one or more other disease nodes; each such connection may have a corresponding conditional probability P. A given conditional probability P(fₖ|dₙ) indicates, for a pair of disease and feature (dₙ, fₖ), the likelihood of the feature fₖ occurring when the disease dₙ observed, and a given conditional probability P(dₙ₁|dₙ₂) indicates, for a pair of disease and other disease (dₙ₁, dₙ₂), the likelihood of the disease dₙ₁ occurring when the disease dₙ₂ is observed. For example, in the depicted embodiment, the following disease-to-feature and disease-to-disease connections and corresponding probabilities are shown (each probability P in this list is intended to also indicate a connection between the two nodes it specifies): P(f₁|d₁), P(f₂|d₁), P(f₃|d₁), P(f₄|d₂), P(d₃|d₂), P(f₆|d₂), P(f₅|d₃), P(dₙ|d₃), P(fₖ|dₙ).

FIG. 1C is a Venn diagram 100C of an illustrative sets definition, according to an embodiment of the invention. The depicted sets are a set of features of a disease d, denoted by F(d), and a set of symptoms, tests, and contexts, denoted by S, and an intersection of the two sets denoted by F(d)∩S.

FIGS. 1B and 1C will be referenced as needed in describing some of the other Figures.

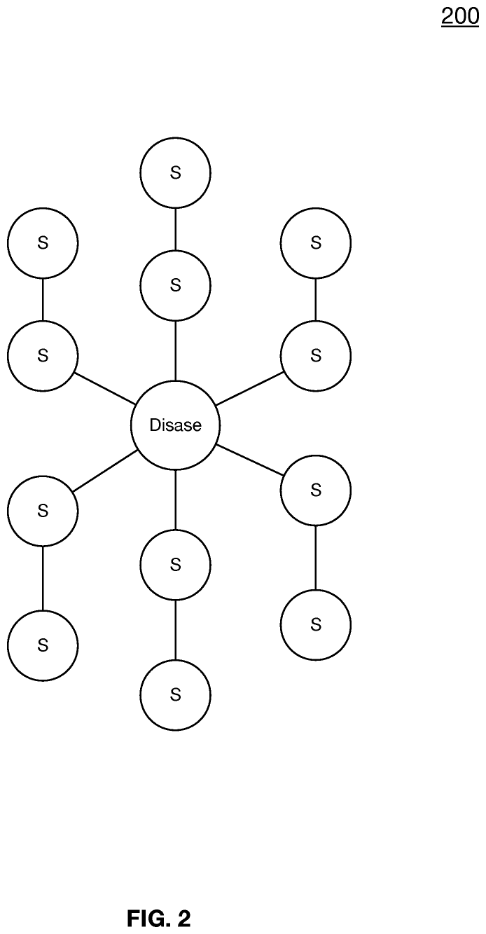

FIG. 2 is a block diagram of an illustrative symptom-disease knowledge graph 200, according to an embodiment of the invention. Symptom-disease knowledge graph 200 is similar to symptom-disease meta-model knowledge graph 100A (FIG. 1), but is illustrated to show, for a given disease (center-node) a set of directly correlated and indirectly correlated symptoms (inner and outer nodes connected to the center node).

Referring now generally to FIGS. 1A, 1B, 1C, and FIG. 2, a challenge in providing machine-guided inquiry recommendation for medical diagnosis (as opposed to a generic, human-practiced medical diagnosis process) is that current systems are unable to generate a most-possible suspected diseases list from the symptom-disease knowledge graph (e.g., symptom-disease meta-model knowledge graph 100A/100B or symptom-disease knowledge graph 200). Note that this is a purely technological problem; i.e., embodiments of the invention do not ask “given a set of symptoms, how does a medical professional come up with a list of most confirmed information”; instead, they ask (and answer) the question of “how does a computer whose data is stored as a symptom-disease knowledge graph generate a list of most possible suspected diseases from confirmed information?”.

For example, consider a patient whose electronic medical record (EMR) for a given visit to a doctor indicates (or confirms) that the patient suffers from a runny nose, a fever, has a body temperature of 38° C. (this may be indicated, for example, via the checks and tests nodes depicted in symptom-disease meta-model knowledge graph 100A/100B), and is 60 years old (this may be indicated, for example, via the patient profile node of symptom-disease meta-model knowledge graph 100A/100B). The technical challenge for the computerized system is how to formulate an effective inquiry question from this data using a technical solution. This challenge may be referred to as the “effective inquiry challenge”.

One approach to machine-guided inquiry recommendation for medical diagnosis employs a term-frequency-inverse document frequency (TF-IDF) approach to calculate probabilities for all diseases in a symptom-disease knowledge graph. TF-IDF is defined by some as a numerical statistic reflecting how significant a word is to a document in a document corpus; and may be used as a weighting factor in information searching and retrieval, text mining, and user modelling. The TF-IDF value of a given word (or entity or concept) increases proportionally to the number of times the word appears in a document, and is offset by the number of documents, in a given corpus, that contain the word. The TF-IDF value is based, in part, on the concept that some words appear more frequently in general; and not necessarily because they are important. For instance, some natural language processes define words such as “the” as unimportant, even though “the” is widely used across English-natural language documents.

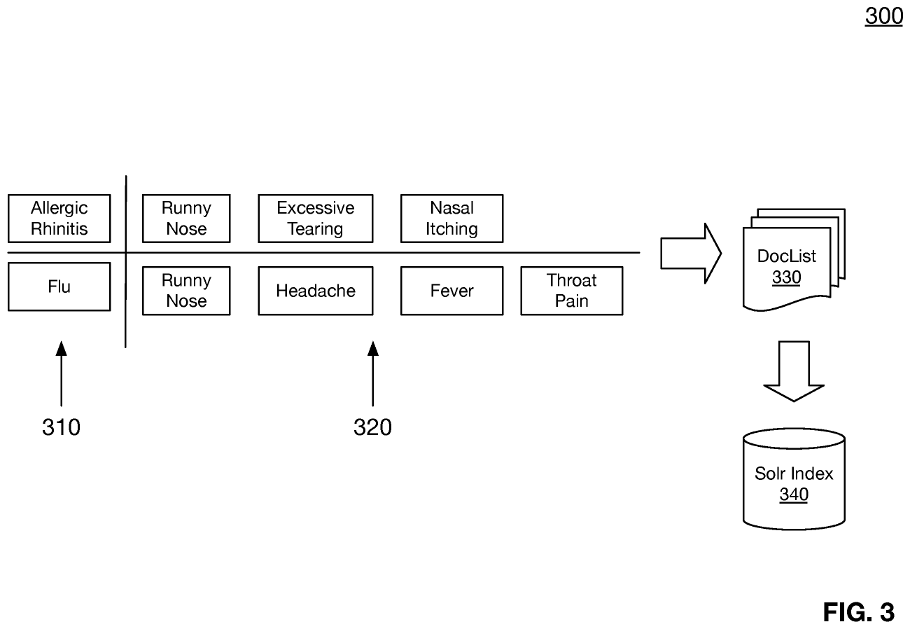

In the prior art, using TF-IDF yields low diagnosis accuracy. FIG. 3 depicts an illustrative process 300 using the TF-IDF approach, where two diseases 310 and their symptoms 320 are evaluated: allergic rhinitis and the flu; the first is associated with a runny nose, excessive tearing, and nasal itching; the second is associated with a runny nose, a headache, a fever, and throat pain. In an example, a query of symptoms, where Q={headache, fever, runny nose}, may be performed using a DocList 330 and a Solr Index 340, to yield a likelihood of 6.78 that Q is indicative of the flu and a likelihood of 2.01 that Q is indicative of allergic rhinitis.

Another approach to machine-guided inquiry recommendation for medical diagnosis employs a Bayesian approach to calculate probabilities for all diseases in a symptom-disease knowledge graph. Using this approach, a set of environmental conditions are mapped to diseases 1−n, which then are mapped to one or more symptoms through a given Bayesian model's modelled mechanism.

Each of the IT-IDF and Bayesian approaches to machine-guided inquiry recommendation for medical diagnosis is limited. Accordingly, embodiments of the invention provide methods and systems that address at least some of these limitations to achieve novel results that are unexpected and non-obvious, and are verified by experimental data. According to an embodiment, a combination of an IT-IDF approach and a Bayesian approach are applied to a symptom-disease knowledge graph generated using a medical guideline corpus of documents. The process includes processing symptom nodes to assign them optimized weights, using the IT-IDF approach; and then applying a Bayesian approach to improve diagnosis accuracy, and to generate a best next inquiry question to enable the diagnosis process to continue based on the Gini index. According to its original definition, the Gini index is a measure of income distribution across income percentiles in a population. Under this definition, a higher Gini index indicates greater inequality, with high income individuals receiving much larger percentages of the total income of the population. Analogically, the Gini index can be applied for measuring the inequality of symptoms for a suspected disease, and the higher Gini index indicates greater inequality of symptoms and a more effective inquiry question.

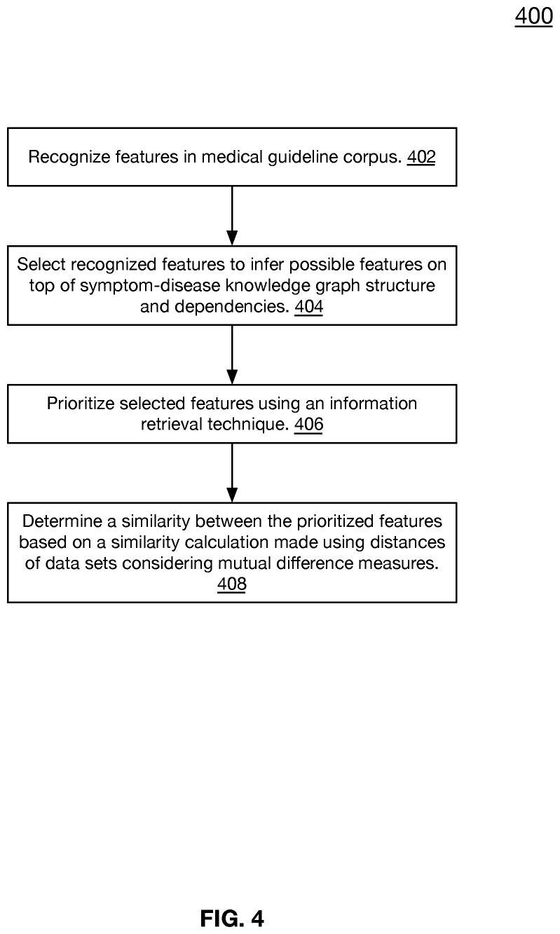

FIG. 4 is a flowchart of a method 400 for calculating a suspected disease using a symptom-disease knowledge graph and a corpus of medical guideline documents (for example symptom-disease knowledge graph 100B of FIG. 1B), according to an embodiment of the invention. Steps of method 400 may be stored as programming instructions on a tangible storage medium of a computer system for machine-guided inquiry recommendation for medical diagnosis, and executed by a processor of the system.

Referring now to FIGS. 1B, 1C, and FIG. 4, at step 402, the computer system recognizes features in the medical guideline corpus by recognizing or allocating nodes in the symptom-disease knowledge graph, using, for example, an object-oriented symptom modelling approach.

At step 404, the computer system selects features recognized at step 402 to infer possible features on top of symptom-disease knowledge graph structure and its dependencies.

At step 406, the computer system prioritizes features selected at step 404 using an information retrieval technique.

At step 408, the computer system determines a similarity between the prioritized features based a similarity calculation made using distances of data sets considering mutual difference measures.

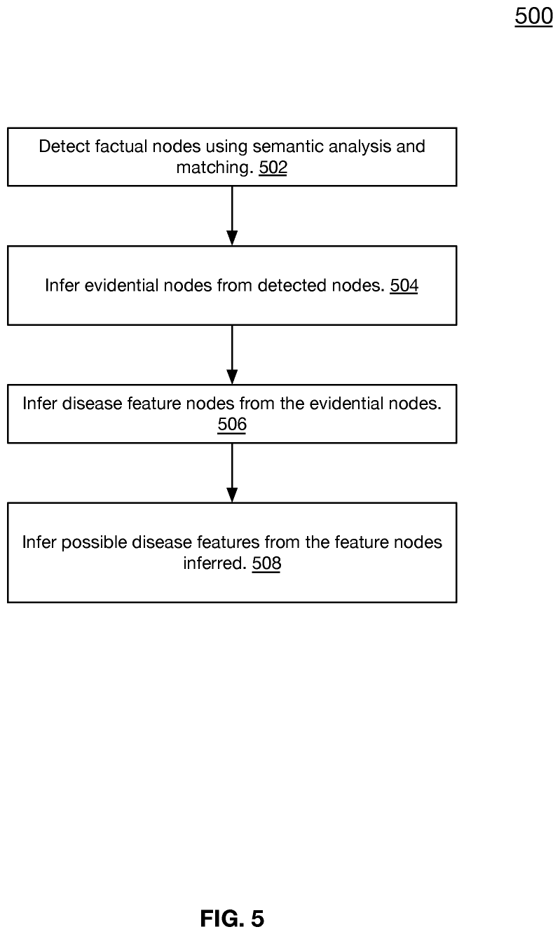

FIG. 5 is a functional flow diagram of a graph-based diagnosis method 500 for calculating a suspected disease using a symptom-disease knowledge graph and a corpus of medical guideline documents (for example symptom-disease knowledge graph 100B of FIG. 1B), according to an embodiment of the invention. Steps of method 500 may be stored as programming instructions on a tangible storage medium of a computer system for machine-guided inquiry recommendation for medical diagnosis, and executed by a processor of the system.

Using data from a set of patient complaint records and a document corpus, graph-based diagnosis process 500 detects factual nodes (for example, symptom nodes, test nodes, context nodes). In one embodiment, graph-based diagnosis process 500 may detect (step 502) factual nodes using semantic analysis and matching. In one embodiment, a confidence that a detected node is a factual node (and therefore should be included in a graph) may be calculated as follows:

Confidence of fact=similarity(input->node) (equation 1)

In one embodiment, graph-based diagnosis process 500 may infer (step 504) evidential nodes from the nodes detected (step 502) in the input data. In one embodiment, the inferring may be performed using reasoning through hyponymy, where a relationship is detected between a generic term (a hypernym) and an instance (hyponym) of the generic term. A hyponym is a word or phrase whose semantic field is more specific that its hypernym. In turn, the semantic field of a hypernym, also known as a superordinate, is broader than the semantic field of a hyponym. In an embodiment, the inferring (step 504) may be based on a confidence value calculated based on conditional probability. For example, the confidence value may be calculated as follows:

Confidence of evidence=Sum(Confidence of fact*P(evidencelfact)) (equation 2)

In one embodiment, graph-based diagnosis process 500 infers (step 506) disease feature nodes from the evidential nodes inferred (step 504). In an embodiment, the inferring (step 506) is performed by reasoning through relation data to feature data, and may use a confidence value calculated based on co-occurrence, as follows:

Confidence of feature=Prod(Confidence of evidence*weight) (equation 3)

In one embodiment, graph-based diagnosis process 500 infers (step 508) possible disease features from the feature nodes inferred (step 506). In an embodiment, the inferring (step 508) is performed by reasoning through relation data to disease data, and may use a confidence value calculated based on feature contributions, as follows:

Confidence of disease=Sum(Confidence of evidence*TF-IDF) (equation 4)

In an embodiment, disease information may be aggregated through hyponymy analysis.

Referring now to FIGS. 1B and 1C, in an embodiment, detecting disease similarity may be performed using a Jaccard Coefficient, where a feature set distance (similarity) is determined using bidirectional difference value(s), according to an embodiment of the invention. The feature set (similarity) may be performed, in one embodiment, using the following equations:

$$\begin{matrix}S ( d ) = freq ( d ) \frac{\vert F ( d ) ⋂ S \vert}{\vert F ( d ) \vert + \vert S \vert - \vert F ( d ) ⋂ S \vert} & ( equation 5.1 ) \\ \vert F ( D ) \vert = {\sum }_{f ∈ d} {w}_{f} & ( equation 5.2 ) \\ \vert F ( d ) ⋂ S \vert = {\sum }_{f ∈ s} {w}_{f} & ( equation 5.3 ) \\ {S}^{′} ( d ) = {freq ( d )}^{*} f ( S ( d ) ) & ( equation 5.4 ) \\ f ( x ) = 1 / {e}^{k ( x - 0.5 )} + 1 & ( equation 5.5 )\end{matrix}$$

In an embodiment, k in equation 5.5 may be equal to (8).

With continued reference to FIGS. 1B and 1C, in an embodiment, calculating weights (for example, those used in equations 3 and 4, above) may be performed using an TF-IDF approach, according to an embodiment of the invention. The weights may be calculated using the following equations:

$$\begin{matrix}\vert F ( d ) \vert = {\sum }_{f ∈ d} {W}_{f} & ( equation 6.1 ) \\ \vert F ( d ) ⋂ S \vert = {\sum }_{f ∈ s} {w}_{f} & ( equation 6.2 ) \\ I D F ( f ) = \frac{\vert D \vert}{f : f ∈ {d}_{i} \vert} & ( equation 6.3 ) \\ \vert F ( d ) \vert = {\sum }_{f ∈ d} {w}_{f} I D F ( f ) & ( equation 6.4 ) \\ \vert F ( d ) ⋂ S \vert = {\sum }_{f ∈ s} {w}_{f} I D F ( f ) & ( equation 6.5 ) \\ {S}^{′} ( d ) = {freq ( d )}^{*} f ( S ( d ) ) & ( equation 6.6 )\end{matrix}$$

With continued reference to FIGS. 1B and 1C, in an embodiment, calculating probabilities (for example, those used in equation 2, above) may be performed using a Bayesian-based approach, according to an embodiment of the invention. The following equations may be used to calculate probabilities:

$$\begin{matrix}S ( d ) = freq ( d ) \frac{\vert F ( d ) ⋂ S \vert}{\vert F ( d ) \vert + \vert S \vert - \vert F ( d ) ⋂ S \vert} & ( equation 7.1 ) \\ p ( d ) = \frac{freq ( d )}{Σ freq ( {d}_{i} )} & ( equation 7.2 ) \\ p ( s \vert d ) \approx \frac{\vert F ( d ) ⋂ S \vert}{\vert F ( d ) \vert} \approx \frac{\vert F ( d ) ⋂ S \vert}{\vert F ( d ) \vert + \vert S \vert - \vert F ( d ) ⋂ S \vert} & ( equation 7.3 ) \\ p ( d \vert s ) = \frac{p ( {d}_{P} ( s \vert d )}{p ( s )} & ( equation 7.4 ) \\ p ( d \vert s ) \approx \frac{S ( d )}{Σ freq ( {d}_{i} ) p ( s )} & ( equation 7.5 ) \\ p ( d \vert s ) \approx \frac{S ( d )}{C} & ( equation 7.6 )\end{matrix}$$

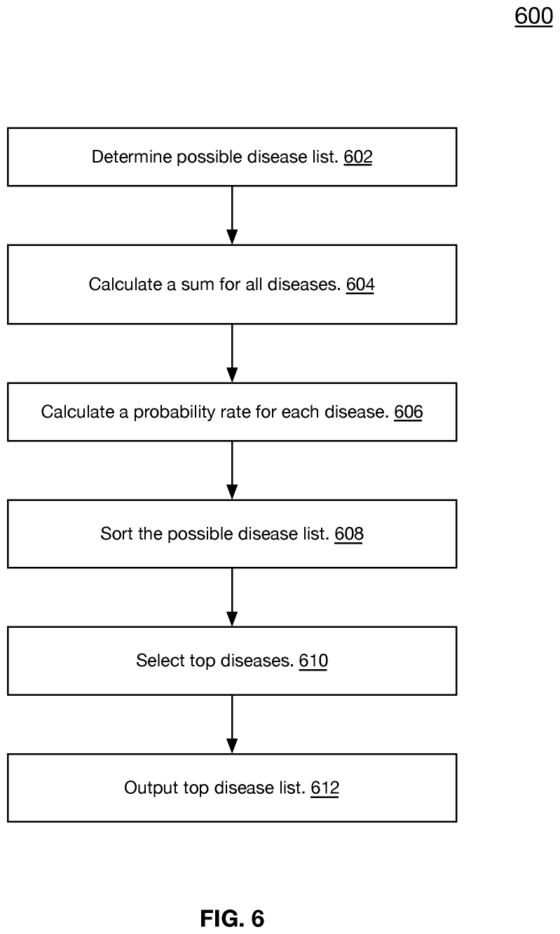

FIG. 6 describes an approach 600 for inferring suspected disease nodes (for example, as described in FIG. 5, above), according to an embodiment of the invention. Steps of method 600 may be stored as programming instructions on a tangible storage medium of a computer system for machine-guided inquiry recommendation for medical diagnosis, and executed by a processor of the system.

Referring now to FIG. 6, the computer system determines (step 602) a possible disease list (PDL) with probabilities (Pi, i=1˜n). The computer system calculates (step 604) a sum (Psum) for all diseases. The computer system calculates (step 606) a probability rate (Ri) for each disease. The computer system sorts (step 608) the PDL by probability rate. The computer system selects (step 610) top diseases whose sum of probability rate is more than a threshold, for example 80%. The computer system outputs (step 612) the top disease list as a suspected disease list. The output may be to a user via a notification including via a graphical user interface or stored an electronic document on a tangible storage medium.

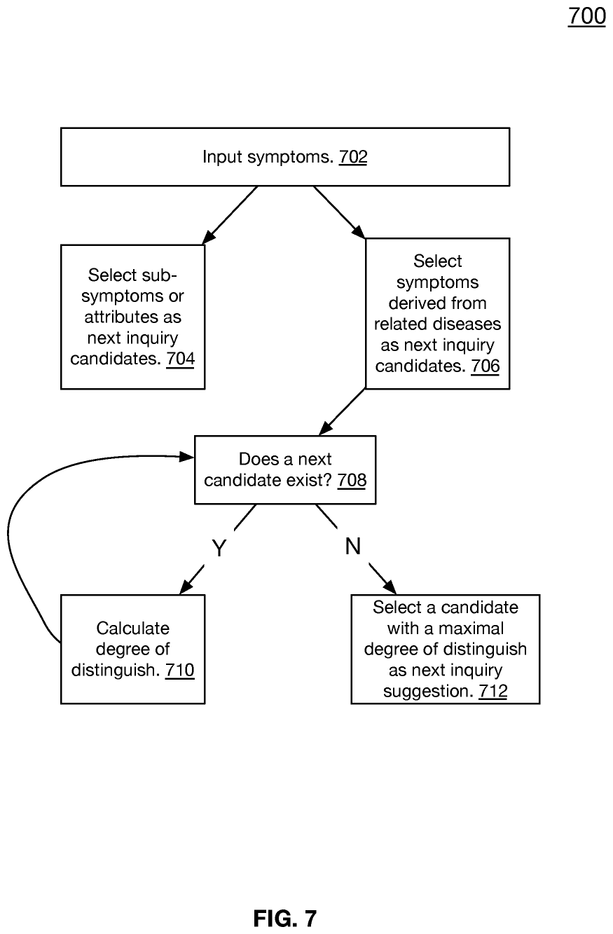

FIG. 7 describes an approach 700 for generating an effective inquiry based on the Gini index, according to an embodiment of the invention. Steps of method 700 may be stored as programming instructions on a tangible storage medium of a computer system for machine-guided inquiry recommendation for medical diagnosis, and executed by a processor of the system.

The computer system inputs (step 702) (or receives) symptoms into its memory. Based on input symptoms, the computer system selects (step 704) sub-symptoms or attributes as next inquiry candidates. Furthermore, based on input symptoms, the computer system selects (step 706) symptoms derived from related diseases as next inquiry candidates. The computer system determines (step 708) whether a next candidate exists. Upon an affirmative determination (YES branch), for a current candidate, the computer system calculates (step 710) its degree of distinguish (DoD); the computer system iterates and performs a next determination (step 708). Upon a negative determination (NO branch), the computer system selects (step 712) a candidate with a maximal DoD as the next inquiry suggestion.

Inventors of the instant invention have tested the various embodiments presented herein, including on 32 common and multiple respiratory system diseases using more than 40 typical cases. Embodiments of the invention provided a top-1 diagnostic accuracy of 61.7% and top-3 diagnostic accuracy of 94.0%, compared to two benchmark prior art methods. The two benchmark methods each provided the following diagnostic accuracy (far less than embodiments of the invention). Benchmark method 1 provided 32.3% accuracy for top-1; 55.9% for top-3; and 70.6 for top-5. Benchmark method 2 provided 41.1% accuracy for top-1; 47.0% for top-3; and 50.0% for top-5. This resulted in the average number of questions being at 13.5 questions compared to 27.7 and 16.6 for the two benchmark methods. Embodiments of the invention provided superior performance.

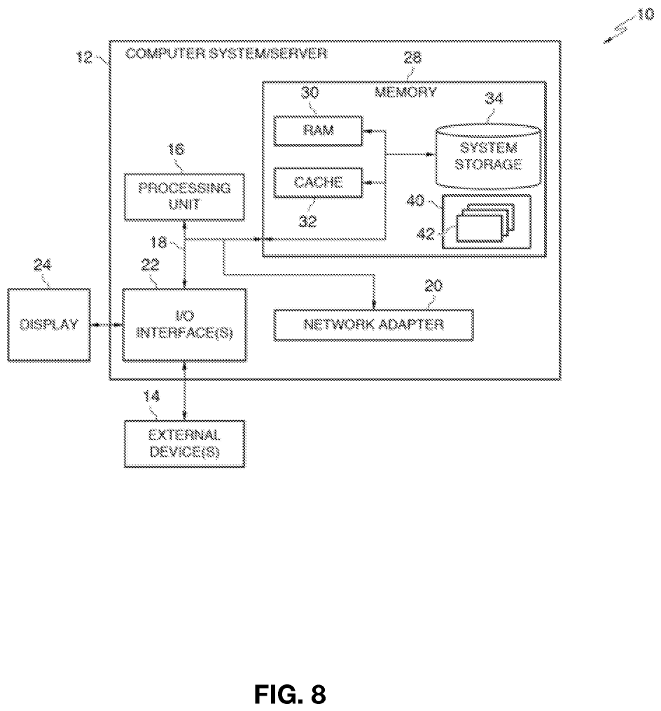

FIG. 8 is a block diagram of an illustrative cloud computing node, according to an embodiment of the invention. Cloud computing node 10 is only one example of a suitable cloud computing node and is not intended to suggest any limitation as to the scope of use or functionality of embodiments of the invention described herein. Regardless, cloud computing node 10 is capable of being implemented and/or performing any of the functionality set forth hereinabove (for example, in connection with FIGS. 1-7, above).

In cloud computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 8, computer system/server 12 in cloud computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

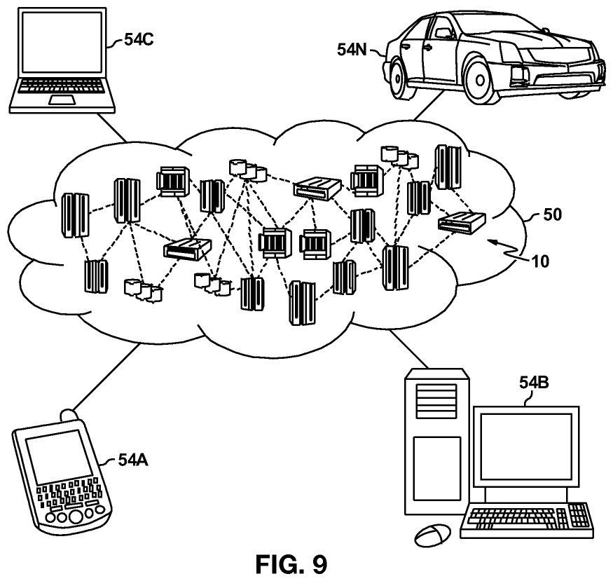

FIG. 9 is a block diagram of a cloud computing environment including the cloud computing node of FIG. 8, according to an embodiment of the invention. Referring now to FIG. 9, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 9 are intended to be illustrative only and that cloud computing nodes 10 and cloud computing environment 50 can communicate with any type of computerized device over any type of network and/or network addressable connection (e.g., using a web browser).

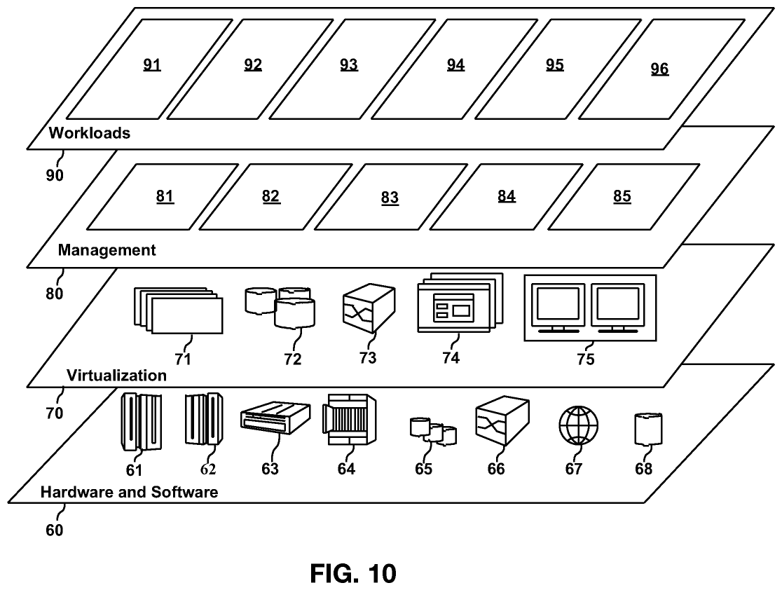

FIG. 10 is a block diagram of functional layers of the cloud computing environment of FIG. 9, according to an embodiment of the invention. Referring now to FIG. 10, a set of functional abstraction layers provided by cloud computing environment 50 is shown. It should be understood in advance that the components, layers, and functions shown in FIG. 10 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and medical inquiry assistance 96, including those described in connection with FIGS. 1-8, above.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for providing a machine-guided inquiry recommendation for medical diagnosis, comprising:
generating a best next inquiry question, for use in a medical diagnosis process, wherein the generating is based on one or more likelihood values that a set of input symptoms exhibits one or more diseases, and wherein the likelihood values are calculated using a knowledge graph data structure generated using one or more electronic medical guideline documents.

2. The method of claim 1, further comprising:
generating the knowledge graph data structure using one or more electronic medical guideline documents.

3. The method of claim 1, wherein calculating the one or more likelihood values comprises:
applying a combined term frequency-inverse document frequency (TF-IDF) process and Bayesian modelling process to one or more diseases in the knowledge graph data structure.

4. The method of claim 3, wherein the applying comprises:
applying the combined TF-IDF process and Bayesian modeling process to obtain an acceptable performance value;
optimizing weight values of symptoms used in the applying; and
applying further Bayesian modelling to improve accuracy of the one or more likelihood values.

5. The method of claim 1, wherein generating a best next inquiry is performed using an index.

6. The method of claim 1, wherein calculating the one or more likelihood values comprises:
performing feature recognition using an object-oriented symptom modeling process;
performing feature selection on recognized features to infer possible features;
performing feature prioritization on selected features using an information retrieval process;
performing a similarity analysis on prioritized features to calculate distance of prioritized features; and
generating a list of suspected diseases based on the similarity analysis.

7. A method for providing a machine-guided inquiry recommendation for medical diagnosis, comprising:
detecting a set of factual nodes in a knowledge graph data structure;
inferring a set of evidential nodes in the set of factual nodes;
inferring a set of disease feature nodes in the set of evidential nodes; and
inferring a set of possible disease nodes in the set of diseases feature nodes.

8. The method of claim 7, wherein detecting a set of factual nodes in a knowledge graph data structure comprises:
measuring, using semantic matching, a similarity between an input text one or more nodes in the knowledge graph data structure, the input text comprising one or more observed symptoms, wherein the measuring yields a confidence score.

9. The method of claim 7, wherein inferring a set of evidential nodes in the set of factual nodes comprises:
identifying one or more candidate evidential nodes among the set of factual nodes, wherein the identifying is based on hyponymy relationships between at least two factual nodes in the set of factual nodes and is further based on a confidence value for each of the one or more candidate evidential nodes;
adding to the set of inferred set of evidential nodes, at least one of the one or more candidate evidential nodes based on its corresponding confidence value, the at least one factual node as an inferred evidential node.

10. The method of claim 7, wherein inferring a set of disease feature nodes in the set of evidential nodes comprises:
determining a set of disease feature nodes in the set of evidential nodes; and
inferring a set of possible disease nodes in the set of diseases feature nodes.

11. A computer program product for providing a machine-guided inquiry recommendation for medical diagnosis, comprising one or more tangible storage media storing programming instructions for execution by one or more processors of one or more computer systems, the programming instructions comprising instructions for:
generating, by the one or more processors, a best next inquiry question, for use in a medical diagnosis process, wherein the generating is based on one or more likelihood values that a set of input symptoms exhibits one or more diseases, and wherein the likelihood values are calculated using a knowledge graph data structure generated using one or more electronic medical guideline documents.

12. The computer programming product of claim 11, further comprising instructions for:
generating, by the one or more processors, the knowledge graph data structure using one or more electronic medical guideline documents.

13. The computer programming product of claim 11, wherein calculating the one or more likelihood values comprises:
applying, by the one or more processors, a combined term frequency-inverse document frequency (TF-IDF) process and Bayesian modelling process to one or more diseases in the knowledge graph data structure.

14. The computer program product of claim 13, wherein the applying comprises:
applying, by the one or more processors, the combined TF-IDF process and Bayesian modeling process to obtain an acceptable performance value;
optimizing, by the one or more processors, weight values of symptoms used in the applying; and
applying, by the one or more processors, further Bayesian modelling to improve accuracy of the one or more likelihood values.

15. The computer programming product of claim 14, wherein generating a best next inquiry is performed using an index.

16. The computer programming product of claim 11, wherein calculating the one or more likelihood values comprises:
performing, by the one or more processors, feature recognition using an object-oriented symptom modeling process;
performing, by the one or more processors, feature selection on recognized features to infer possible features;
performing, by the one or more processors, feature prioritization on selected features using an information retrieval process;
performing, by the one or more processors, a similarity analysis on prioritized features to calculate distance of prioritized features; and
generating, by the one or more processors, a list of suspected diseases based on the similarity analysis.
